(19) United States
(12) Patent Application Publication
(10) Pub. No.: US 2026/0013820 A1
(43) Pub. Date: Jan. 15, 2026

(54) Method and Device for Automated Planning of a Radiological Examination

(71) Applicant: Siemens Healthineers AG, Forchheim (DE)

(72) Inventors: Julian Wohlers, Erlangen (DE); Stephan Nufer, Erlangen (DE)

(73) Assignee: Siemens Healthineers AG, Forchheim (DE)

(21) Appl. No.: 19/265,018

(22) Filed: Jul. 10, 2025

(30) Foreign Application Priority Data
Jul. 12, 2024 (DE) 10 2024 206 606.0

(51) Int. Cl. A61B 6/00; A61B 6/46; G16H 20/40; G16H 50/20

(52) U.S. Cl. CPC A61B 6/5294; A61B 6/467; A61B 6/545; G16H 20/40; G16H 50/20

(57) ABSTRACT

The disclosure relates to automated planning of a radiological examination, comprising providing information about a pending examination of a patient from a radiology information system) and providing at least one RIS comment relating to the examination. The automated planning also includes comparing, in an automated manner, character strings in the RIS comment with names of examination programs in a specified examination list, and selecting a number of examination programs for these character strings based on the comparison. Finally, the selected examination programs are output.

### CROSS-REFERENCE TO RELATED APPLICATIONS

The present application claims priority to and the benefit of Germany patent application no. DE 10 2024 206 606.0, filed on Jul. 12, 2024, the contents of which are incorporated herein by reference in their entirety.

### TECHNICAL FIELD

The disclosure relates to a method and apparatus for automated planning of a radiological examination and to a medical imaging system.

### BACKGROUND

There is currently no standardized, technology-assisted end-to-end process for defining the optimum imaging program for an image modality, for instance a CT scanner or a magnetic resonance tomography facility, to scan the necessary images of a patient on which a radiologist can then base a diagnosis.

Currently, the referring physician's instruction, which is input to a scheduling system, for example a radiology information system (“RIS”), is interpreted to acquire an image. This interpretation is performed after the appointment has been arranged for the patient. Such scheduling systems typically have what are known as “attributes,” in which can be found information about an examination or about the patient to be examined.

An examination is then carried out on the basis of this information, for example on the basis of the “Requested intervention” attribute of the RIS and, if available, additional information such as the medical issue, the admission diagnosis, or other relevant information. Radiologists or sometimes even radiographers define the image acquisition protocol, according to which the patient is to be scanned.

Unfortunately, a special RIS comment field is often used for the input, into which information is then entered that should actually be entered into other attributes (for example input fields, information units, or other input instances), or some necessary additions or amendments are added into this RIS comment field, for instance “MR standard brain+TOF.”

The technician who is operating the imaging system concerned for the examination must then interpret the comment provided via the RIS and manually select the relevant examination program (or “program” for short). Then, the patient is registered and any extra amendments and additions to the examination program are made.

Often, the program that the chief technician or the radiologist mentions in the RIS comment is very similar or even identical to the program name in the MR program tree. The requested technique, however, is not suitable on its own for guiding program selection because it is usually worded too broadly.

Currently, the following procedure is often adopted:
the order reaches the hospital or the radiology institute;
the job and, if available, further information such as the admission diagnosis, the clinical issue, etc. are interpreted by a radiologist or a technician;
the program is prescribed on the basis of the information available with the order (see above), SOPs (“standard operating procedures”), available medical records and questions answered by the patient;
information about the examination program to be used for the scan is provided to the radiographer at the scanner via the RIS;
technologists interpret the information in the RIS and choose and assign the program and/or steps manually from the available file structure of the programs on the MR system;
the technician adds specific individual steps to the MR-scan queue from the step library of the examination program;
the technician amends the settings for the examination program, for example program strategies, decisions in the program, etc., based on the information provided, the observed condition of the patient (for example breath-holding capacity, movement sensitivity, etc.) and on the SOPs, or based on his scan experience.

### SUMMARY

This leads to the problem that radiology departments need a qualified workforce for this purpose who interpret the inquiry and know the examination programs that can best answer this inquiry. In addition, the procedure is prone to errors in creating the request or in interpreting the request. The procedure is also time-consuming, and it is laborious manually finding the desired screening program in the available program structure. Should additional steps be required that are not part of the standard screening program, it can take a long time to find and add these program steps manually.

It is thus an object of the present disclosure to define a method and apparatus for automated planning of a radiological examination, and also a medical imaging system which avoid the disadvantages described above and e.g. make possible automated (advance) planning of programs for an examination.

This object is achieved by the various embodiments described herein, including the claims.

A method according to the disclosure is used for automated planning of a radiological examination. It comprises the following steps:
providing information about a pending examination of a patient from a radiology information system (“RIS”), wherein at least one RIS comment relating to the examination is provided;
comparing, in an automated manner, character strings in the RIS comment with names of examination programs in a specified examination list, and selecting a number of examination programs for these character strings based on the comparison;
outputting the selected examination programs.

The method is used for automated planning of a radiological examination. What is meant by this is that a number of examination programs are selected in an automated manner from information in an RIS about a pending examination of a patient. The chosen examination programs can then be used directly for an examination or form a sub-selection from which a human or a machine can then make a definite selection for an examination. The problem here is that specific examination programs are often not described in the RIS in the correct attribute (but in the comment), or that too few or ambiguous details are given in the correct attribute and specific details are given in the comment.

In short, from a perfectly completed entry in an RIS, automated planning of a radiological examination may normally be derived directly from the relevant attributes in the RIS. If, however, the entry is not perfectly worded, the method according to the disclosure makes it possible to perform automated planning nonetheless.

First, information about a pending examination of a patient may be provided from an RIS. This might be, for example, an entry about the pending examination of the patient that has been input into the RIS in order to schedule the examination. This information may comprise at least one RIS comment, which is contained in this information. It is quite possible that the information contains yet more information in other attributes in the RIS, for instance the “Requested procedure” attribute.

When this information is available, an automated comparison is performed on character strings in the RIS comment with names of examination programs in a specified examination list. These names are mostly universal in a machine pool of similar machines, and optionally be defined in advance. In a simple embodiment, all the available examination programs, and e.g. also path names in a directory tree in which these examination programs exist, are entered into the examination list. For example, the name of an examination program may be “Standard brain,” but there may also be an examination program with the name “Standard” in the “Brain” folder. This is known in advance, however, so that the examination list can be set up accordingly.

A comparison can be made e.g. by a direct comparison of character strings in the RIS comment with character strings in the examination list. It is also possible, however, to seek first a group of character strings that are synonymous with a character string in the RIS comment, and then to compare the synonyms together with the original character string with entries in the examination list. A semantic comparison is also possible by means of advanced algorithms, for instance trained adaptive models.

Based on this comparison, a number of examination programs are selected for these character strings in the RIS comment and the selected examination programs are output. For example, in the case that the entry “Standard brain” can be found in the RIS comment, the corresponding entry is sought in the list. Should a plurality of examination programs exist with the name “Standard”, they can all be output for subsequent further selection, or a targeted search be subsequently made for which of these names can be found in the “Brain” folder, and this examination program is output.

In short, the method according to the disclosure makes use of the available information from the RIS and analyzes the RIS comment field (and other attributes if applicable). Then, a semantic similarity is determined between the comment in the RIS comment field and the programs (e.g. name and path name) available in the program trees on the system. Then, the examination program or the program path with the greatest similarity to the RIS comment is selected automatically.

The text field may be examined here for possible operators, for instance “AND” or “+”. Such operators are often used to concatenate examination programs. Program steps and additional steps are concatenated according to the operators in the RIS comment field. Settings from the main program (for example breath-holding settings) are used for the parameterization of the concatenated steps.

If a plurality of possible examination programs are identified, the list of the possible examination programs can be presented, and a user can manually select the programs and the steps that are meant to be concatenated and used for the registration. Another implementation can go so far as extracting the information provided and categorizing it into relevant information blocks.

In an embodiment, a user interface may provide (e.g. in a configuration window) information that indicates the information that was used to make the selection of a certain program. This has the advantage that the selection of the examination programs is transparent for administrators of the system, for instance chief technicians or service engineers. This information may be checked and opportunities given to weight specific information blocks to optimize the selection.

Information such as the required contrast agent may e.g. be extracted together with physical information about the patient, for instance weight and test results, and passed to the selected examination program to parameterize the examination program automatically.

An apparatus according to the disclosure is used for automated planning of a radiological examination. It comprises the following components:
a data interface designed to receive information about a pending examination of a patient from a radiology information system, wherein at least one RIS comment relating to the examination is provided;
a comparison unit designed to compare, in an automated manner, character strings in the RIS comment with names of examination programs in a specified examination list, and to select a number of examination programs for these character strings based on the comparison;
a data interface designed to output the selected examination programs.

The function of the components of the apparatus has already been described above. The apparatus may e.g. be designed to perform a method according to the disclosure.

A medical imaging system according to the disclosure may comprise for instance an MRT system or a CT system, and comprises an apparatus according to the disclosure and/or is designed to perform the method according to the disclosure.

The embodiments of the disclosure may be realized e.g. in the form of a computer unit with suitable software. For this purpose, the computer unit can comprise, for example, one or more interacting microprocessors or the like. For instance, embodiments of the disclosure may be realized in the form of suitable software program parts in the computer unit. An implementation largely in software has the advantage that even computer units already in use can be easily upgraded by a software or firmware update to work in the manner according to the disclosure. In this respect, the object is also achieved by a corresponding computer program product comprising a computer program, which can be loaded directly into a storage device of a computer unit, and which contains program segments in order to perform all the steps of the method according to the disclosure when the program is executed in the computer unit. Said computer program product may comprise, in addition to the computer program if applicable, extra elements such as for example documentation and/or extra components, including hardware components such as for example hardware keys (dongles etc.) for using the software.

For transfer to the computer unit and/or for storage on, or in, the computer unit, a computer-readable medium, for instance a memory stick, a hard disk, or any other portable or permanently installed data storage medium may be used, on which are stored the program segments of the computer program, which program segments can be downloaded and executed by a computer unit.

Further, advantageous embodiments and developments of the disclosure are described herein in the claims and in the description, where the embodiments in one category may also be developed in a similar way to the embodiments in another category, and e.g. individual features of different exemplary embodiments or variants can also be combined to create new exemplary embodiments or variants.

According to an embodiment of the method, in a radiology information system a radiological examination is specified or chosen, e.g. the next one pending for a specified examination system. Then, an RIS comment field, and for instance also a field for the input of a desired examination is sought for this examination in the radiology information system. In a further step, it is checked whether the relevant field (the RIS comment field or, if applicable, the field for the input of a desired examination) contains character strings. These character strings are then provided as an RIS comment. For example, say the entry “MR head” is the desired examination that has been input; the entry “unexplained headaches, Standard with CA” is the entry in the RIS comment. It can then be inferred therefrom that an MRT examination of the head is meant to take place, and it can be inferred from the details in the RIS comment that for this is meant to be used the standard program with contrast agent. Examination programs for a standard MR examination of the head with contrast agent are then picked from the examination list. If only one has been found, this is suggested to the user. If a plurality of possible ones are found, the user can be provided with a selection.

If, in addition to the RIS comment, the entries in further attributes are used for the comparison, a hierarchy can be defined. As an example, the field for inputting a desired examination can be taken into account first. If the result from a subsequent comparison is just a single hit in the examination list, the step of the automated comparison of character strings in the RIS comment is skipped. If, however, the result is a plurality of options or no option, the automated comparison of character strings in the RIS comment with the entries in the examination list is performed.

If all the information is insufficient, no examination program is selected.

In an embodiment, as part of the comparison of the character strings, a semantic similarity between a character string in the RIS comment and the name of an examination program in the examination list is ascertained qualitatively by reference to a similarity measure and/or a distance metric. A number of examination programs are then selected that have the greatest similarity. In an embodiment, an algorithm automatically selects the program and, if applicable, also the program path with the greatest similarity to the RIS comment. Various distance metrics are known that may be used in the context of computational linguistics (e.g. natural language processing (NLP)), and also established similarity measures that can be used for this purpose.

According to an embodiment, as part of the comparison of the character strings, these are first classified into different semantic blocks, e.g. into a semantic block which
relates to a body part to be examined,
relates to a diagnosis to be verified,
relates to symptoms,
relates to a clinical issue,
relates to a patient condition (for example “cooperative”, “uncooperative”, “hearing impaired”, “can hold breath for #s max.”, “speaks the language . . . ”. Such attributes may also be used later for further parameterization of the examination program (for example whether breath-hold commands or voice commands are output in the patient's language),
relates to contraindications (for example implant status, contrast agent compatibility, possible contrast agent intolerances),
relates to certain medications that are meant to be used, for example Buscopan or Adenosin, or
relates to heuristic information in the database of the algorithm.

It may be checked whether one of these character strings has a similarity to a character string of a corresponding semantic block of a previous examination. If so, a number of examination programs of this previous examination are selected. For example, if a patient A with similar information has been given program X, it is likely that this program X may also be suitable for patient B with very similar medical conditions. Many radiology departments try to work in a standardized way. Therefore, many comments are similar or identical, which will result in the same program selection. Usually, the name of the program is stated directly or indirectly in the RIS comment.

According to an embodiment of the method, the character strings in the RIS comment are examined for logical operators that suggest e.g. concatenations or links. To this end, a parser for instance (a program which scans the character strings) examines the RIS comment for specified character strings, e.g. character strings that are indicative of a logical “AND” or a logical “NOT”, for instance the characters “AND”, “UND”, “ET” or “+”, or “NOT”, “NICHT” or “NON”, or a comma, a semicolon or simply a space character.

In an embodiment, examination programs that have been selected using character strings directly before and after such an operator are linked to one another in accordance with the operator, and e.g. arranged for execution directly after one another. Such operators are used for concatenation. Program steps and additional steps are concatenated according to the operators in the RIS comment field. Settings from the main program (for example breath-holding settings) are used for the parameterization of the concatenated steps. One example is the entry “Tumor screening with CA”, where the word “with” is interpreted as a logical “AND” in the sense of 1. The tumor screening program AND 2. contrast agent administration.

In an embodiment, in the case in which a plurality of examination programs have been selected for a character string, these are presented to a user for selection. For example, a list of specified examination programs is filtered on the basis of the parsing results, and the result of this filtering is displayed to a user, who can manually select the desired examination programs.

In an embodiment, the examination programs are present in the form of groups, e.g. in the form of program trees, in the examination list. When a group is selected, then all the group elements, e.g. when a branch is selected, all dependent branches, may be selected as the examination programs.

In an embodiment, an examination program may be selected by means of a plurality of character strings, wherein one of the character strings identifies the program, and a number of other character strings identify program paths, branches of a tree structure or groups or subgroups. For example, it is checked whether a word or a series of words could describe a program and whether such a program actually exists.

For example, the entry “Standard brain+TOF” means that a standard brain examination (i.e. the standard brain program) is meant to be used for an examination, but the entry additionally (“+”) requests a further image series with a time of flight (TOF) measurement, which depicts the vascular tree of the brain well. The method then identifies the “Standard brain” examination program by a comparison with the examination list and decides, since “TOF” is not part of “Standard brain”, that TOF is a further examination program that is meant to be employed additionally (*+*). Users often try to express by way of such a combining operator (logical AND) that they want the scan to include further series in addition to the standard program.

According to an embodiment, in addition to an output of an examination program or a group of examination programs, that number of character strings are output that have been used to select this examination program or a group of examination programs. For example, a user interface provides in a configuration window information that indicates the information that was used to make the selection of a certain program, so that the selection is transparent for a user. This information can be checked and opportunities are given to weight specific information blocks higher in order to optimize the selection.

Additional further information may for instance be derived from the RIS comment, and e.g. also from other attributes in the radiology information system. This may include for instance information about a required contrast agent, and/or the patient, e.g. height, weight, age, gender or pre-existing conditions, and/or test results, and/or machine parameters. Such details are contained in further attributes of an RIS entry, but are also mentioned in the RIS comment. There are also, for example, other private or public attributes. For example, “pregnant” is an individual attribute. There is also, however, the attribute “Alert”, in which is entered information about allergies or other warnings. For instance, age, weight, height and gender of a patient are individual attributes that are always retrieved in the registration. Test results, for instance creatinine or GFR (globular filtration rate), are laboratory parameters that can be used to decide whether an examination is with or without contrast agent. This information may e.g. be output together with the selected examination programs, and/or the selected examination programs are pre-configured automatically with this information.

According to an embodiment, after the output of the selected examination programs, the examination programs that are employed for a radiological examination in question are determined. This information is then used as part of the method for a subsequent automated comparison of character strings in the RIS comment. This information may e.g. be used to train a machine learning model, which may constitutes the ground truth. Alternatively or additionally, this information may be linked to the character strings.

In an embodiment, a search is also made for the “Requested procedure” attribute in addition to the RIS comment. Information about which examination program is meant to be selected can often be found here. If this information is present, a check is carried out for which examination program was selected for other patients most often in the past for the character string in question in the RIS comment. The “Requested procedure” attribute is often kept very general, for instance “MR head”, signifying no more than that an MRT examination of the head is meant to be made. The RIS comment often contains further information, however, which gives specifics, for instance that an oncological follow-up examination of the head is involved or “Unexplained headaches” or a “Multiple Sclerosis+contrast agent” examination. As a result, completely different MRT acquisitions of the head arise from “MR head”. Therefore, the current implementation of automatically assigning “Requested procedure” to examination program is in itself not sufficiently robust and accurate.

An apparatus comprises a configuration unit, which is designed additionally to derive further information from the RIS comment, e.g. information about a required contrast agent, and/or the patient, e.g. height, weight, age, gender or pre-existing conditions, and/or test results, and/or machine parameters, and to pre-configure a number of selected examination programs automatically with this information.

In an embodiment, AI-based methods (AI: “artificial intelligence”) may be implemented to perform any of the methods according to the disclosure. Artificial intelligence is based on the principle of machine-based learning, and is usually performed by an adaptable algorithm that has been suitably trained. The term “machine learning” is often used for machine-based learning, and also includes the principle of “deep learning”.

Components of the disclosure may be available e.g. as a “Cloud service”. Such a Cloud service is used to process data, e.g. by means of artificial intelligence, but can also be a service based on conventional algorithms, or a service in which humans perform an analysis in the background. In general, a Cloud service (also abbreviated below to the “Cloud”) is an IT infrastructure in which is provided storage space or computing power and/or application software, for example, via a network. The communication between the user and the Cloud is achieved by means of data interfaces and/or data transfer protocols. In the present case, the Cloud service may for instance provide both computing power and application software.

As part of any of the methods according to the disclosure, data that is obtained may be provided via the network to the Cloud service. This comprises a computing system, which normally does not comprise the local computer of the user. The method can be realized in this case by means of a command constellation in the network. The data computed in the Cloud may be later sent back to the local computer of the user via the network.

### BRIEF DESCRIPTION OF THE DRAWINGS

The disclosure is described again below in greater detail using exemplary embodiments and with reference to the accompanying figures. Identical components are denoted by the same reference numbers in the various figures, which are generally not shown to scale and in which:

FIG. 1 illustrates a schematic representation of an example magnetic resonance tomography system having an apparatus according to an exemplary embodiment of the disclosure; and

FIG. 2 illustrates a method for an example automated planning of a radiological examination according to an exemplary embodiment of the disclosure.

### DETAILED DESCRIPTION OF THE DISCLOSURE

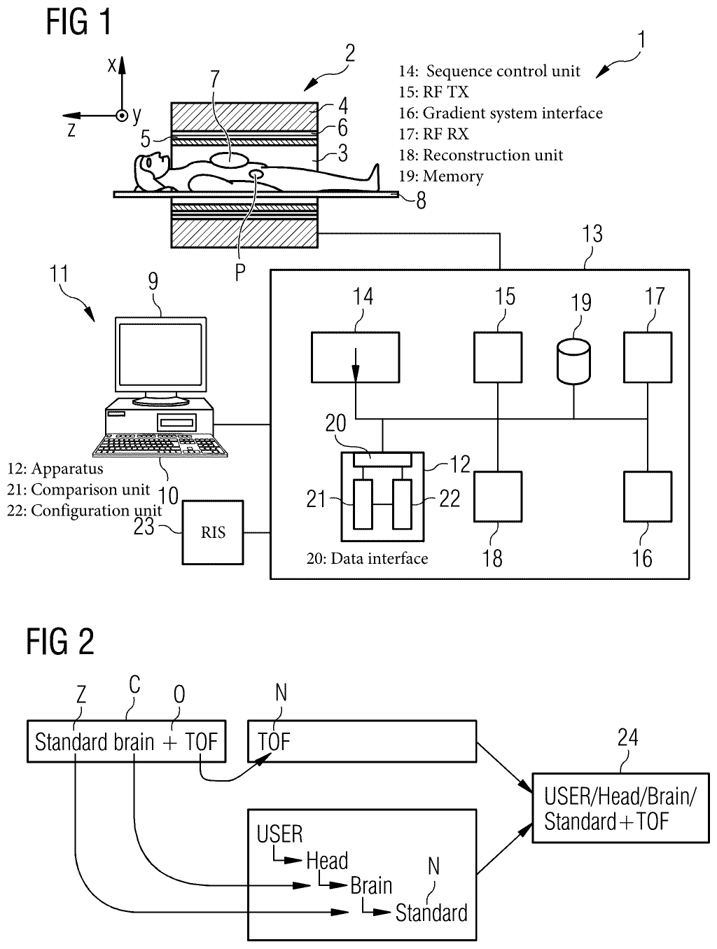

FIG. 1 illustrates a schematic representation of an example magnetic resonance tomography system having an apparatus according to an exemplary embodiment of the disclosure. More specifically, FIG. 1 shows a highly simplified representation of a magnetic resonance tomography system 1. It comprises the actual magnetic resonance scanner 2 containing an examination space 3 or patient tunnel, into which is positioned, on a couch 8, a patient or person under examination, in whose body is located the actual object under examination P.

The magnetic resonance scanner 2 is equipped in the usual manner with a main magnetic field system 4, a gradient system 6, and also an RF transmit antenna system 5 and an RF receive antenna system 7. In the exemplary embodiment shown, the RF transmit antenna system 5 is a body coil that is fixed in the magnetic resonance scanner 2, whereas the RF receive antenna system 7 consists of local coils (represented here just by a single local coil) to be arranged on the patient or person under examination. In principle, however, the body coil can also be used as the RF receive antenna system, and the local coils can be used as the RF transmit antenna system, provided these coils can each be switched into different operating modes. The main magnetic field system 4 is designed here in the usual manner to generate a main magnetic field in the longitudinal direction of the patient, i.e. along the longitudinal axis of the magnetic resonance scanner 2, which axis extends in the z direction. As is customary, the gradient system 6 comprises individually controllable gradient coils to be able to switch gradients in the x, y or z direction independently of one another.

The magnetic resonance tomography system shown here is a full-body system containing a patient tunnel, into which a patient can be placed completely. In principle, however, the disclosure can also be used on other magnetic resonance tomography systems, for instance having a C-shaped enclosure that is open at the side. Any suitable acquisitions of the subject under examination P can be produced.

The magnetic resonance tomography system 1 also comprises a control device 13 (also referred to herein as a central control device, controller, or control circuitry), which is used to control the MR system 1. This control device 13 comprises a sequence control unit (also referred to herein as a sequence controller or sequence control circuitry) 14. This may e.g. be used to control the succession of radiofrequency pulses (RF pulses) and gradient pulses according to a selected pulse sequence or according to a succession of a plurality of pulse sequences for acquiring a plurality of slices in the acquisition region within a measurement session. Said pulse sequence can be specified and parameterized in a measurement protocol or control protocol, for example. Different control protocols for different measurements or measurement sessions are typically stored in a memory 19, and can be selected (and possibly modified if required) by operating personnel, and then used to perform the measurement.

The examination region can be defined by selected pulse sequences or the positioning of the aforementioned RF receive antenna system 7.

For the output of the individual RF pulses of a pulse sequence, the control device 13 comprises a radiofrequency transmit device 15, which generates and amplifies the RF pulses, which it feeds into the RF transmit antenna system 5 via a suitable interface (not shown in detail). The control device 13 may also comprise a gradient system interface 16 for controlling the gradient coils of the gradient system 6 to switch the gradient pulses suitably according to the specified pulse sequence. The diffusion gradient pulses and spoiler gradient pulses may be applied via this gradient system interface 16. The sequence control unit 14 may communicate in any suitable manner, for example by sending out sequence control data, with the radiofrequency transmit device 15 and the gradient system interface 16 to implement the pulse sequence.

The control device 13 also has a radiofrequency receive device 17 (likewise communicating with the sequence control unit 14 in a suitable manner) to receive magnetic resonance signals within the readout window defined by the pulse sequence in a coordinated manner by means of the RF receive antenna system 7, and hence to acquire the raw data.

A reconstruction unit (also referred to herein as a reconstruction controller or reconstruction circuitry) 18 receives here the acquired raw data and reconstructs the magnetic resonance image data therefrom. Again, this reconstruction is usually performed on the basis of parameters, which can be specified in the measurement protocol or control protocol concerned. This image data can then be stored in a memory 19, for example.

The principles of how suitable raw data can be acquired by applying RF pulses and switching gradient pulses, and how MR images or parameter maps can be reconstructed therefrom, are known fundamentally to a person skilled in the art and therefore are not explained further here.

The control device 13 is also connected to an RIS 23, which is used to organize the course of examinations by the MR system 1.

For the automated planning of a radiological examination, the control device 13 comprises an apparatus 12 according to the disclosure comprising a data interface 20, a comparison unit 21 (also referred to herein as comparison circuitry or a comparator) and a configuration unit (also referred to herein as configuration circuitry or a configurator) 22.

The data interface 20 is used to receive information about a pending examination of a patient P from a radiology information system 23, wherein at least one RIS comment C relating to the examination is provided (see FIG. 2). In addition, the data interface 20 is used to output the selected examination programs 24.

The comparison unit 21 is used to compare, in an automated manner, character strings Z in the RIS comment C with names N of examination programs 24 in a specified examination list, and to select a number of examination programs 24 for these character strings Z based on the comparison.

The configuration unit 22 is designed additionally to derive further information from the RIS comment C, e.g. information about a required contrast agent, the patient P, for instance height, weight, age, gender, pre-existing conditions, etc., about test results and/or machine parameters. A number of selected examination programs 24 can thereby be pre-configured automatically with this information.

FIG. 2 illustrates a method for an example automated planning of a radiological examination according to an exemplary embodiment of the disclosure. More specifically, FIG. 2 shows a method for automated planning of a radiological examination.

On the left, information about a pending examination of a patient P is provided from a radiology information system, which information is in the form of an RIS comment C relating to the examination. The entry in the comment reads: “Standard brain+TOF”.

In the center, character strings in this entry, i.e. “Standard”, “brain”, “+”, and “TOF” are compared with names N of examination programs 24 in a specified examination list U. The examination list U comprises in this example the names N of the examination programs 24 and the names N of the paths. The “+” character is interpreted here as the linking operator O, signifying that “Standard brain” and “TOF” must both be employed for the examination.

On the right is shown the selection of the examination programs 24 for these character strings Z. The “Standard” examination program was found in the “Brain” path, which belongs to the “USER/Head” branch. The TOF examination program was found in another folder. This combination of examination programs 24 is then output.

Finally, it should be reiterated that the disclosure described in detail above involves exemplary embodiments, which can be modified by a person skilled in the art in many different ways without departing from the scope of the disclosure. In addition, the use of the indefinite article “a” or “an” does not rule out the possibility of there also being more than one of the features concerned. Likewise, terms such as “unit” do not exclude the possibility that the components in question consist of a plurality of interacting sub-components, which may also be spatially distributed if applicable. The term “a number of” shall be interpreted as “at least one”. Independent of the grammatical term usage, individuals with male, female or other gender identities are included within the term.

Additionally, the various components described herein may be referred to as “units.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such units, as applicable and relevant, may alternatively be referred to herein as “circuitry,” “controllers,” “processors,” or “processing circuitry,” or alternatively as noted herein.

What is claimed is:

1. A method for automated planning of a radiological examination, comprising:
providing, via a radiology information system, information about a pending radiological examination of a patient, the information including a radiology information system (RIS) comment relating to the radiological examination;
automatically comparing character strings in the RIS comment with names of radiological examination programs in a predetermined radiological examination list;
selecting, based on the comparison, one or more of the radiological examination programs corresponding to the character strings;
outputting the selected one or more of the radiological examination programs to facilitate execution of one or more of the one or more selected radiological examination programs to perform the radiological examination.

2. The method as claimed in claim 1, further comprising:
selecting a next pending radiological examination for an examination system;
identifying, in the radiology information system, an RIS comment field and a field for an input of the pending radiological examination; and
verifying whether (i) the field for the input of the pending radiological examination contains character strings, and (ii) the character strings are provided as the RIS comment.

3. The method as claimed in claim 1, further comprising:
automatically comparing the character strings by qualitatively ascertaining a semantic similarity between a character string in the RIS comment and a name of a radiological examination program in the predetermined radiological examination list by referencing a similarity measure and/or a distance metric; and
selecting, as the one or more radiological examination programs, those radiological examination programs having the greatest similarity.

4. The method as claimed in claim 1, further comprising:
automatically comparing the character strings by classifying the character strings into different semantic blocks that relate to:
a body part to be examined;
a diagnosis to be verified;
symptoms;
a clinical issue;
the patient;
contraindications;
medications; or
heuristic information in a database, and
verifying whether (i) one of the character strings has a similarity to another character string of a corresponding semantic block of a previous radiological examination, and (ii) a number of radiological examination programs of the previous radiological examination are selected.

5. The method as claimed in claim 1, further comprising:
processing logical operators in the character strings in the RIS comment corresponding to concatenations or links by parsing the RIS comment for character strings comprising a logical AND operator or a logical NOT operator;
linking radiological examination programs to one another that have been selected using character strings directly before and directly after the logical AND operator or the logical NOT operator in accordance with the respective logical AND operator or the logical NOT operator; and
arranging the linked radiological examination programs to be executed directly after one another.

6. The method as claimed in claim 1, further comprising:
presenting, to a user for selection, the one or more radiological examination programs that were selected based on the character strings.

7. The method as claimed in claim 1, wherein the one or more radiological examination programs are stored as groups in program trees in the predetermined radiological examination list, and further comprising:
upon selecting a branch of the program trees, each group element from among respective dependent branches are selected as the one or more radiological examination programs; and
selecting the one or more examination programs via a plurality of character strings,
wherein one of the plurality of character strings identifies the program, and a number of other ones of the plurality of character strings identify respective program paths, branches of the program tree, groups, or subgroups.

8. The method as claimed in claim 1, further comprising:
outputting a number of character strings that were used to select one of the one or more radiological examination programs or a group of the one or more radiological examination programs.

9. The method as claimed in claim 1, further comprising:
deriving further information from (i) the RIS comment, and (ii) other attributes in the radiology information system comprising supplemental information with respect to one or more of:
a required contrast agent;
the patient;
the patient's height, weight, age, gender, and/or pre-existing conditions;
test results; and
machine parameters, and
wherein (i) the supplemental information is output with the selected one or more radiological examination programs, and/or (ii) the selected one or more radiological examination programs are automatically preconfigured with the supplemental information.

10. The method as claimed in claim 1, further comprising:
after outputting the selected one or more radiological examination programs, determining the one or more radiological examination programs to be executed for the radiological examination; and
utilizing further information with respect to the determined one or more radiological examination programs for a subsequent automated comparison of character strings in the RIS comment; and
training a machine learning model and/or linking the further information to the character strings.

11. An apparatus for automated planning of a radiological examination, comprising:
a data interface configured to receive, from a radiology information system, information about a pending radiological examination of a patient, the information including a radiology information system (RIS) comment relating to the radiological examination;
comparison circuitry configured to automatically compare character strings in the RIS comment with names of radiological examination programs in a predetermined radiological examination list, and to select, based on the comparison, one or more of the radiological examination programs corresponding to the character strings,
wherein the data interface is configured to output the selected one or more of the radiological examination programs, to facilitate execution of one or more of the one or more selected radiological examination programs to perform the radiological examination.

12. The apparatus as claimed in claim 11, further comprising:
configuration circuitry configured to determine further information from the RIS comment with respect to one or more of:
a required contrast agent;
the patient,
the patient's height, weight, age, gender or pre-existing conditions,
test results, and
machine parameters,
and to automatically pre-configure a number of selected examination programs with the further information.

13. The apparatus as claimed in claim 11, wherein the apparatus comprises part of a magnetic resonance tomography (MRT) system or a computed tomography (CT) system.

14. A computer-readable storage medium having instructions stored thereon that, when executed by computing circuitry, cause the computing circuitry to automatically plan and execute a radiological examination by:
providing, via a radiology information system, information about a pending radiological examination of a patient, the information including a radiology information system (RIS) comment relating to the radiological examination;
automatically comparing character strings in the RIS comment with names of radiological examination programs in a predetermined radiological examination list;
selecting, based on the comparison, one or more of the radiological examination programs corresponding to the character strings;
outputting the selected one or more of the radiological examination programs; and
executing one or more of the one or more selected radiological examination programs to perform the radiological examination.
